(19) United States
(12) Patent Application Publication
(10) Pub. No.: US 2022/0218049 A1
(43) Pub. Date: Jul. 14, 2022

(54) Thumb Protector Device for a Cast and Method

(71) Applicant: Justin Stephan Roth, Bend, OR (US)

(72) Inventor: Justin Stephan Roth, Bend, OR (US)

(21) Appl. No.: 17/575,491

(22) Filed: Jan. 13, 2022

Related U.S. Application Data
(60) Provisional application No. 63/136,758, filed on Jan. 13, 2021.

(51) Int. Cl. A41D 13/08

(52) U.S. Cl. CPC A41D 13/087

(57) ABSTRACT

A thumb protector is provided which is engageable with a cast formed upon the hand of a patient. A body of the thumb protector has a ring portion defining an opening for a thumb of a patient wearing the cast. A surrounding portion is positionable to a sandwiched engagement between fabric wrapped around the hand and forearm of the patient and the arm and hand. An engagement point of the ring portion to the surrounding portion forms an anchor for the wrapped fabric to prevent slippage thereof once the cast is formed.

This application claims priority to U.S. Provisional patent application Ser. No. 63/136,758, filed on Jan. 13, 2021, which is incorporated herein in its entirety by this reference thereto.

### FIELD OF THE INVENTION

The disclosed device generally concerns a device and method for positioning a cast on the hand of a patient. More particularly, it relates to a thumb protecting device and method for positioning thereof, on casts employed on the hand of a patient after an injury or following orthopedic surgery.

### BACKGROUND OF THE INVENTION

An orthopedic cast, or simply a cast, is a shell, conventionally formed from plaster, and more modernly, formed from fiberglass and resin, which encases a limb to stabilize it and hold it in an anatomically correct position during healing. Such casts are conventionally employed on patients for the duration of time required for healing.

Plaster casts conventionally consist of a cotton bandage that has been combined with plaster of paris, which hardens after being wetted. Modernly, casts are formed of synthetic materials, such as knitted fiberglass bandages impregnated with polyurethane, sometimes bandages of thermoplastic. Such synthetic casts cure faster than those formed of plaster and are much lighter.

However, patient discomfort, over the long duration a cast is positioned on the patient, continues to be a significant problem. This is especially true of fiberglass upper extremity casts positioned to cover the hand. Such casts conventionally will encase the palm of the hand and allow for extension of the fingers from an open end, and the thumb from a side-positioned opening.

With the thumb communicating through this side opening on the cast over a long duration, skin irritation occurs in the first web space located between the thumb and index finger. Additional irritation is known to occur around the circumference of the thumb where it communicates against the surface of the cast surrounding it.

The forgoing examples of casts in positions upon the hand of a patient following injury or surgery, and issues regarding such, are intended to be illustrative and not exclusive and they do not imply any limitations on the invention described and claimed herein. Various other limitations of the related art are known or will become apparent to those skilled in the art upon a reading and understanding of the specification below and the accompanying drawings.

### SUMMARY OF THE INVENTION

The device and method, herein disclosed, provide for a significant enhancement in comfort for patients who must wear casts on their hands and forearm subsequent to an injury or surgery.

A thumb protector and padding device is provided, which is engageable to or with a conventional fiberglass and plaster cast, during the conventional process of forming such a cast on the hand of a patient. Consequently, no special equipment or procedures are required for use, and the thumb protector can be operatively engaged to a cast during the formation process.

Conventionally, a cast is formed upon a patient requiring it in a sequence. First, a stockinette is placed upon and around the area of the body of the patient, which will be covered by the cast. With this stockinette properly positioned, a layer of under-cast padding made of cotton or another soft material such as WEBRIL, is rolled on in a covering layer, to further protect the skin.

With the stockinette and under-cast padding layers in place, fiberglass strips from a roll are moistened and wrapped around the padding layer. Once properly positioned and cured, this exterior fiberglass layer cures to form the cast.

In the system herein, a cast is formed upon the patient in a similar conventional wrapping sequence. However, a thumb protector is positioned during the formation of the case to form a padded annular opening for protection of the thumb communicating through it.

In the sequence using the thumb protector herein, the stockinette is placed upon and around the area of the body of the patient, which will be covered by the cast. With this stockinette properly placed, the thumb protector is positioned such that a padded opening surrounds the base of the thumb, and a flexible planar body portion of the thumb protector surrounds the opening. In some cases the thumb protector may be positioned upon the hand of the user prior to positioning of the stockinette. In either fashion, a padded annular opening is provided to the patient to protect their thumb from the surrounding formed cast.

Once the thumb protector is properly placed, either over or under a stockinette or similar conventional fabric layer, the standard cast formation continues, and the layer of padding such as WEBRIL is rolled on to cover both the stockinette and any padding layer of the body portion surrounding the thumb opening of the thumb protector. With the layer of stockinette and the layer of padding and the thumb protection in place, the step of positioning the fiberglass strips can be accomplished. Once the fiberglass layer is wetted and cured, the cast is formed, with the flexible body portion of the thumb protector secured within the layers, and the padded opening providing protection to the thumb projecting therefrom.

In a particularly preferred mode of the thumb protector device and method herein the thumb protector and flexible body portion surrounding the padded annular thumb opening are formed of polymeric material such as, for example, silicone or polyethylene. Preferably, the flexible body portion may have ridges and/or recesses or combinations of both formed therein. These ridges and/or recesses form an uneven surface into which the layers of fabric material forming the cast will naturally settle. This of the overlain cast layers therein, forms a stabilizing engagement between the thumb protector device and the layers forming the cast, which resists movement of the thumb protector device over time. Such is most preferred to maintain the thumb protector in place and from folding or wrinkling underneath the cast, which would cause discomfort to the patient.

Additionally preferred are the positioning of a polymeric annular ring portion of the thumb protector which surrounds the opening through which the thumb of the patient communicates. The ring is preferably formed of a polymeric material which is soft but durable and of a low durometer, such as silicone, polyethylene, or a polyurethane gel elastomer, or another soft but durable polymeric material. The ring may be provided in various sizes and may be formed such that it will work for both left and right-handed casts. So positioned, this combination of a padding ring and flexible surrounding body serves to protect the thumb from chafing and irritation, as well as protecting the first web of the hand.

This ring portion forms an annular circular pad surrounding the opening for the thumb, which provides a curved pliable contact surface surrounding the entire thumb. Additionally, the curved ring portion extends with a circumference to form a curved recess where the ring portion connects to the flexible body portion. This curved recess provides an anchor point for any padding wrapped around the body portion and any fiberglass or conventional hardening cast material. This anchor point thus prevents the padding and/or cast material from riding up over the ring portion during and after cast formation, thus insuring the patient will enjoy a padded separation of the thumb and skin adjacent thereto, from the rigid cast.

With respect to the above description, before explaining at least one preferred embodiment of the thumb protecting hand case device and method herein in detail, it is to be understood that the invention is not limited in its application to the details of construction and to the arrangement of the components in the following description or illustrated in the drawings. The invention herein described and shown is capable of other embodiments and of being practiced and carried out in various other ways by those skilled in the art upon reading this disclosure. Also, it is to be understood that the phraseology and terminology employed herein are for the purpose of description and should not be regarded as limiting.

As such, those skilled in the art will appreciate that the conception upon which this disclosure is based may readily be utilized as a basis for designing of other thumb and surrounding area protective devices for hand casts, and for carrying out the several purposes of the present disclosed device. It is important, therefore, that the claims be regarded as including such equivalent construction and methodology insofar as they do not depart from the spirit and scope of the present invention.

As used in the claims to describe the various inventive aspects and embodiments, “comprising” means including, but not limited to, whatever follows the word “comprising.” Thus, use of the term “comprising” indicates that the listed elements are required or mandatory, but that other elements are optional and may or may not be present. By “consisting of” is meant including, and limited to, whatever follows the phrase “consisting of.” Thus, the phrase “consisting of” indicates that the listed elements are required or mandatory, and that no other elements may be present. By “consisting essentially of” is meant including any elements listed after the phrase, and limited to other elements that do not interfere with or contribute to the activity or action specified in the disclosure for the listed elements. Thus, the phrase “consisting essentially of” indicates that the listed elements are required or mandatory, but that other elements are optional and may or may not be present depending upon whether or not they affect the activity or action of the listed elements. The term “substantially” when employed herein, means plus or minus ten percent unless otherwise designated in a different range.

It is an object of the present invention to provide a device and method for positioning a more comfortable cast upon the hand of a patient which protects the thumb and first web of the hand of the patient from chafing and irritation from the surrounding cast.

It is a further object to provide such a cast which employs a thumb padding component which securely locates a soft protective ring or bumper surrounding the opening for communication of the thumb of a patient therethrough, to provide a significant comfort enhancement for the patient.

It is another object of this invention to provide such a thumb padding component which is configured with position stabilizers to maintain it in a proper positioning relative to the surrounding cast.

Another object of this invention is the provision of a pliable ring portion surrounding the thumb of the patient, which also is configured to provide an anchor point to prevent padding and fiberglass or other cast material from riding up and over the ring after the cast is formed.

It is a further object of this invention to provide such a thumb padding device which is easily employable in conventional fiberglass and plaster casts, to encourage widespread use.

These and other objects, features, and advantages of the present device and method for forming a padded passage for thumb communication therethrough, as well as the advantages thereof over existing prior art, which will become apparent from the description to follow, are accomplished by the improvements described in this specification and hereinafter described in the following detailed description which fully discloses the invention, but should not be considered as placing limitations thereon.

### BRIEF DESCRIPTION OF DRAWING FIGURES

The accompanying drawings of FIGS. 1-10, which are incorporated herein and form a part of the specification, illustrate some, but not the only or exclusive examples of embodiments and/or features of the disclosed cast thumb protector and method herein. It is intended that the embodiments and figures disclosed herein are to be considered illustrative of the invention herein, rather than limiting in any fashion.

In the drawings:

FIG. 1 depicts a perspective view of the thumb protector device herein operatively positioned on the hand of a patient, showing the soft ring surrounding the opening for the thumb and the flexible surrounding body portion surrounding the soft ring, which is configured to form a curved contact against the curved skin surface of both sides of the hand.

FIG. 2 depicts a view of the thumb protector device herein operatively positioned, as viewed from the palm side of the hand, showing the surrounding body portion forming against the surface of the hand or against the surface of a fabric stockinette, if positioned on the hand prior to the device.

FIG. 3 depicts a sectional view of a cast formed with the thumb protector device herein and showing the soft ring portion of the device surrounded by the flexible body portion where the flexible body portion has recesses and/or ridges therein, where the device is positioned on top of the stockinette layer and underneath the subsequently applied layers.

FIG. 4 shows a formed cast upon the hand of the patient and shows the soft annular ring portion surrounding the opening through which the thumb of the patient projects and anchoring any padding and/or cast material to prevent an override of the ring and maintain the padded ring in position.

FIG. 5 shows a perspective side view of the thumb protector device herein showing the annular ring portion forming a recess therearound at its intersection to the flexible body portion to provide an anchor point for padding and/or cast material wrapped around the device during cast formation. Also shown are the ridges and recesses therebetween formed in a radial pattern surrounding the annular ring.

FIG. 6 shows the thumb protector device herein in a mode having projections extending from the upper surface which are sized to project into gaps between the fibers of padding and/or casting material wrapped therearound.

FIG. 7 is an overhead view of the device herein showing the annular ring portion surrounding and defining the thumb opening and the curved surface entirely surrounding that opening and also showing radially projecting ridges and recesses.

FIG. 8 depicts an overhead view of a mode of the device herein which has a plurality of circular ridges surrounding the ring and opening.

FIG. 9 shows an overhead view of a mode of the device herein showing the curved pliable annular ring portion surrounding the thumb opening and showing curved recesses formed at four corners of the flexible body portion, which allow for adjustment to the curve of the hand and avoid wrinkling.

FIG. 10 depicts the device herein, as in FIG. 9, showing the anchor point formed at the intersection of the annular pliable ring portion to the flexible body portion and showing radially positioned projections as well as traverse running projections.

### DETAILED DESCRIPTION OF THE INVENTION

In this description, the directional prepositions of up, upwardly, down, downwardly, front, back, top, upper, bottom, lower, left, right, first, second, and other such terms refer to the device as it is oriented and appears in the drawings and all such terms are used for convenience only and such are not intended to be limiting or to imply that the device has to be used or positioned in any particular orientation.

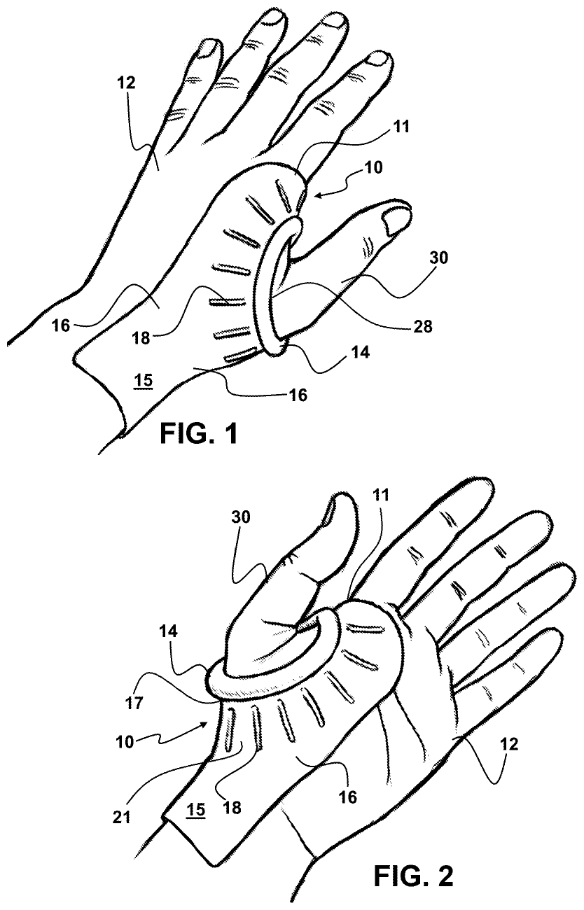

Now referring to drawings in FIGS. 1-10, wherein similar components are identified by like reference numerals, there is seen in FIGS. 1 and 2 opposing perspective views of the thumb protector device 10 herein. As shown, the device 10 is operatively positioned on the hand 12 of a patient in an as-used positioning. By as-used positioning is meant that the device 10 is positioned upon the hand 12 of the patient with the thumb 30 of the patient projecting through the opening 28 and a first side surface of the flexible surrounding portion 16 positioned in contact against the skin of the user or against a fabric such as a stockinette, if employed.

The device 10 features a body 11 formed of an annular ring portion 14 which is engaged to and surrounded by a generally planar and flexible surrounding portion 16. Both the ring portion 14 and the surrounding portion 16 are preferably formed of a soft polymeric material, such as for example only, silicone or polyethylene. However, other plastic, blastomeric, or polymeric materials having a low durometer, and having a flexibility to conform to the contours of the hand of a patient may be employed.

Further, the body 11 of the device 10 may be formed in unitary structure in a single component or it may be formed to a unitary structure by co-molding or other means for manufacture where the ring portion 14 may be formed of a differing material to that of the surrounding portion 16. For example only, the ring portion 14 might be formed of a polyurethane gel elastomer which will easily conform to the contours of the thumb and surrounding hand especially well and provide exceptional padding, and the surrounding body portion 16 might be formed of silicone or polyethylene or the like.

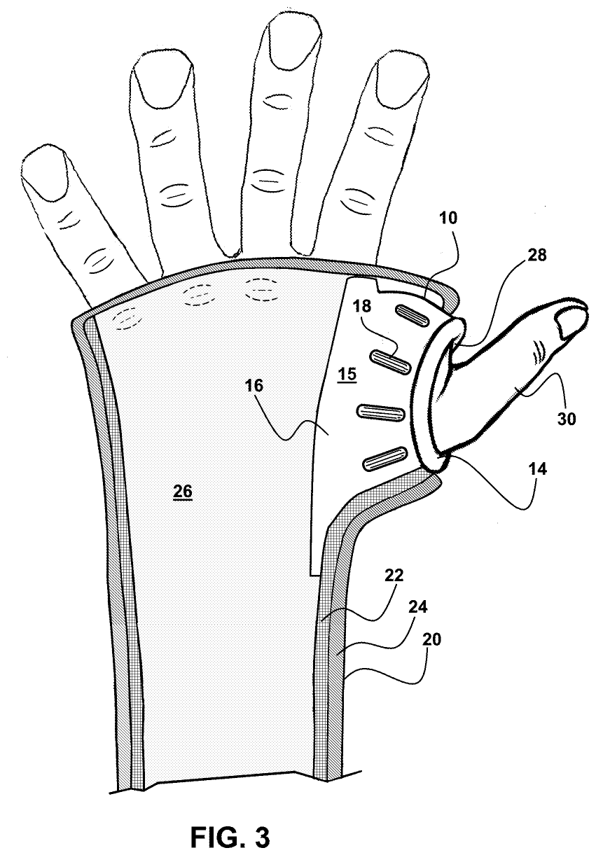

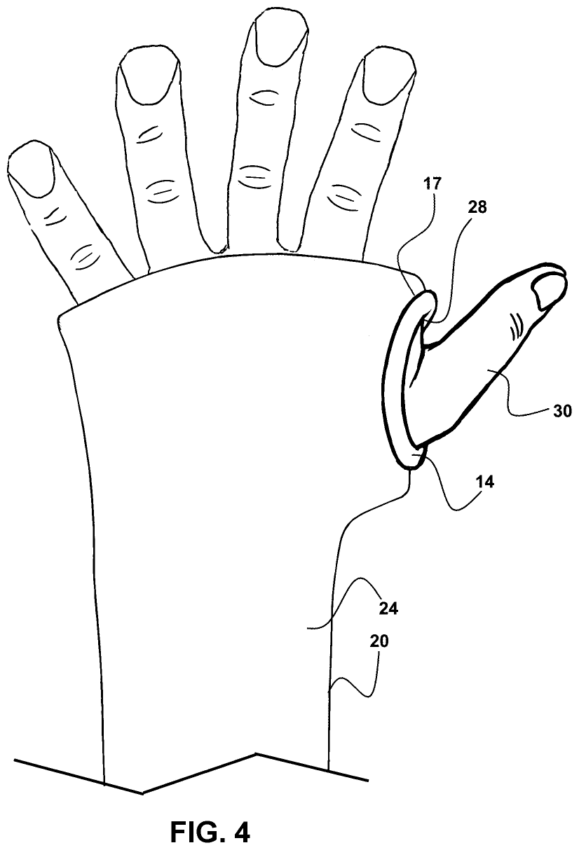

Additionally shown in FIGS. 1-3 and 5-10 are position stabilizers 18 which are positioned upon the upper or second surface 15 of the surrounding portion 16 of the body of the device 10. While the device 10 will significantly improve the comfort of a cast positioned on a patient without such position stabilizers 18, such are particularly preferred to enhance the engagement of the device 10 to the fabric and other layers forming the cast 20 (FIGS. 3-4). Such position stabilizers 18 are shown extending for example as projections 19 rising from the second surface 15 which form recesses 21 therebetween. As shown, the projections 19 are in a plurality forming a radial pattern surrounding the ring portion 14 of the device 10.

Currently, the position stabilizers 18 are formed as such projections 19 and/or recesses 21 upon the second surface 15 of the flexible surrounding portion 16 of the device 10. Such will cause a significant enhancement of the frictional engagement between the surrounding portion 16 of the device 10 around the ring portion 14, and the under-cast padding layer 22 (FIG. 3) of the formed cast 20 and/or the overlain casting hardening material. Once the fiberglass layer 24 (FIG. 3) or similar wrapped hardening cast material is overlain upon the under-cast padding layer 22 and is cured, the position stabilizers 18 serve to maintain the device 10 in the proper as-used positioning on the hand 12 of the patient, such as is shown in FIGS. 1-2. This serves to prevent wrinkling and dismounting which can cause discomfort to the hand of the patient over time.

Shown in FIG. 3 is a sectional view of a formed cast 20 having the thumb protector device 10 herein and showing the pliable and soft ring portion 14 of the body of the device 10 and the flexible surrounding portion 16 thereof. As noted, the surrounding portion 16 is formed of a low durometer material and is substantially planar, such that it will easily fold and conform to the contours of the hand of the patient.

As shown in FIG. 3, the device 10 is positioned atop the stockinette layer 26 of the formed cast 20, and underneath the under-cast padding 22 and the cured fiberglass layer 24. However, in some cases the device 10 may be positioned with the first side surface 13 in contact with the skin of the hand 12 of the patient and with the stockinette layer 26 wrapped therearound in contact with the second surface 15.

Where the preferred position stabilizers 18 are included, upon the surrounding portion 16 of the body of the device 10, they are contacted on either the first side surface 13 or second side surface 15 of the surrounding portion 16 by the stockinette layer 26 and on the opposing side of the surrounding portion 16 by the under-cast padding 22 in a sandwiched engagement therebetween. As noted, such a positioning of the position stabilizers 18 where included, enhances the fixed positioning of the device 10 within the cast 20 for the duration it is worn by a patient. Thus, folding and wrinkling of the surrounding portion 16 is also prevented which would otherwise cause patient discomfort.

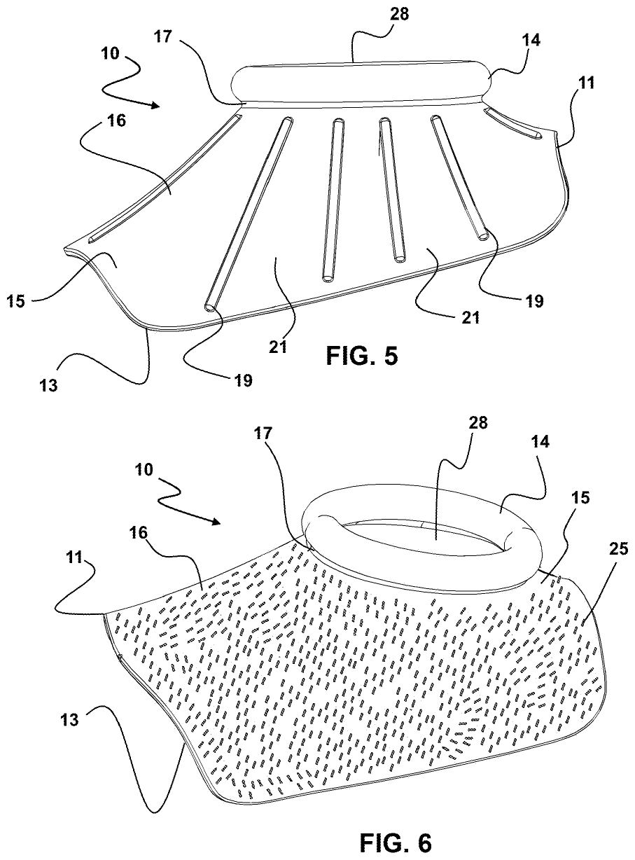

Also, as noted, FIG. 4 depicts the formed cast 20 operatively positioned in an as-used positioning upon the hand 12 of the patient. As can be seen, the ring portion 14 of the device 10 is engaged in a contact at an anchor point 17 formed therearound by an overhang of the curved surface of the ring portion 14 adjacent the intersection of the surface of the ring portion 14 with the surrounding portion 16 (FIG. 5). This anchors the engagement of the ring portion 14 with the sidewall of the cast 20.

As shown, the ring portion 14 has a curved exterior surface which defines an annular opening 28 therein, configured for communication of the thumb 30 of the patient therethrough. So positioned, the ring portion 14 provides a soft curved circumferential edge to the opening 28, while the surrounding portion 16, as shown in FIG. 3, helps protect the hand and first web adjacent the thumb 30 from chafing and other irritation.

In FIG. 5 is shown a perspective side view of the thumb protector device 10 herein showing the first side surface 13 configured to fold toward and conform to the curved surfaces of the hand of the patient surrounding their thumb 30. At the intersection of the annular ring portion 14 with the second surface 15, is defined a recess 17 therearound by the overhanging curved exterior surface of the ring portion 14. In use, this recess 17 area defines an anchor point around the entire ring portion 14 for padding and/or cast material wrapped around the device 10 during cast formation. This anchoring helps maintain the ring portion 14 and the device 10 in firm positioning to the cast and in place for the duration of the casting.

Also shown, rising from the second surface 15 are the position stabilizers 18. As shown, the position stabilizers 18 are formed by projections 19 or ridges and recesses 21 therebetween formed in a radial pattern surrounding the annular ring portion 14. When padding material and/or fiberglass 24 or similar cast-hardening material are wrapped upon the second surface 15 and anchored in the anchoring point 17 such forms into the recesses 21 and over the projections 19 to hold the device 10 especially secure in place.

Shown in FIG. 6 is the thumb protector device 10 herein in a mode having small diameter projections 25 extending from the upper or second surface 15 to respective distal ends. These projections 25 are sized in diameter such that they will project into gaps between the fibers of the woven padding and/or casting material wrapped therearound. Thus, each such projection 25 forms an anchor point into openings formed between the warp and fill yarns of any overlain fabric material and helps maintain the device 10 and especially the surrounding portion 16 in place. Where conventional woven gauze or padding material is employed, such openings, for example, may be between 0.012 and 0.235 inches across. As such, the diameter of the projections 25 would be substantially between 0.013 and 0.234 inches. However, those skilled in the art will realize this diameter can be adjusted so long as the distal ends of the projections 25 project into the openings on the fabric employed.

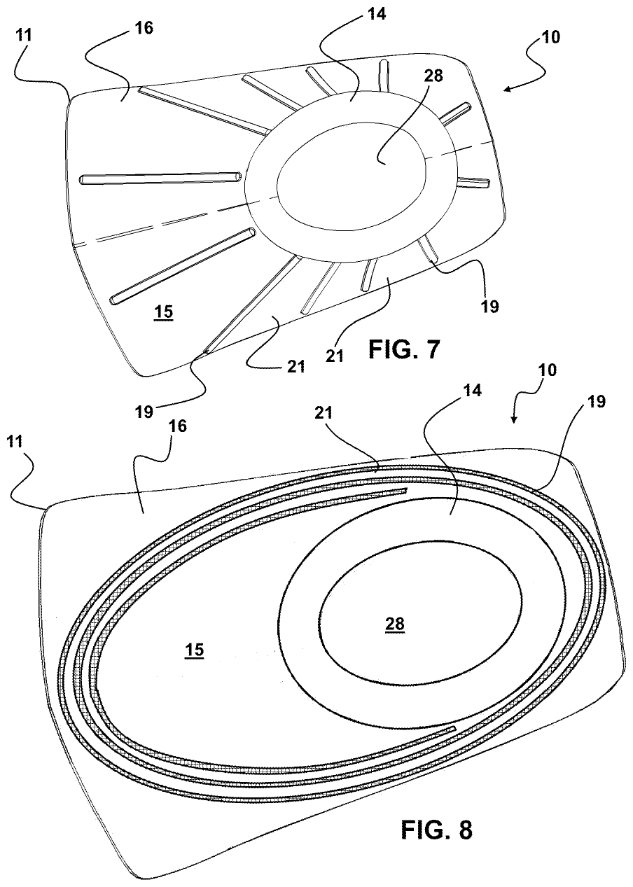

Depicted in FIG. 7 is an overhead view of the device 10 herein showing the annular ring portion 14 surrounding and defining the thumb opening 28 and the curved surface of the exterior of the ring portion 14 entirely surrounding that opening 28. Also shown are radially positioned projections 19 or ridges and recesses 21 defined therebetween.

In FIG. 8 is shown an overhead view of a mode of the device 10 herein wherein the plurality of projections 19 forming ridges and recesses 21 are formed as plurality of circular projections 19 extending above the second surface 15. This forms a plurality of circular recesses 21 therebetween.

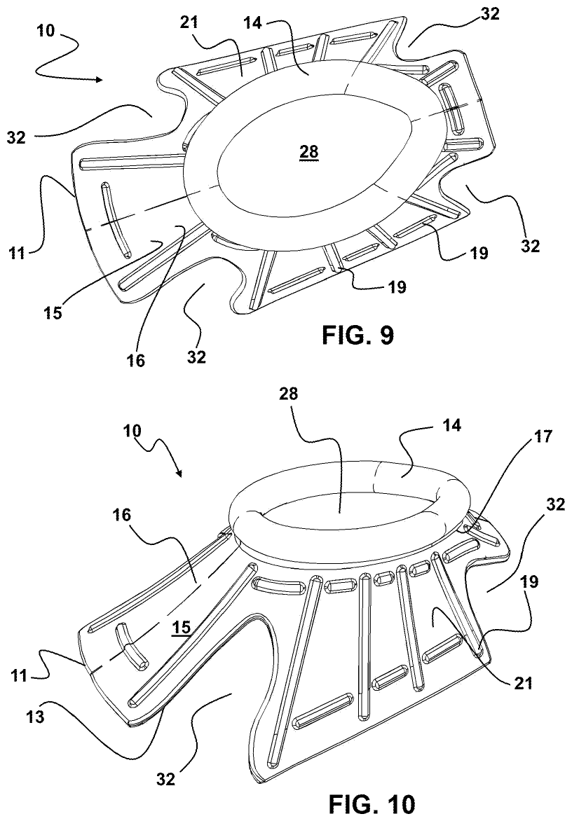

In FIG. 9 is shown an overhead view of a mode of the device 10 herein showing the curved pliable annular ring portion 14 surrounding and providing a curved contact surface around the thumb opening 28. Also included are a plurality of curved indentations 32 positioned at four locations around the circumference of the surrounding portion 16. While shown as four indentations 32 it is anticipated there can be more or less than four. The indentations 32 allow for the portions of the surrounding portion 16 of the body 11 therebetween to move toward each other as the surrounding portion 16 is placed upon the hand of the user into the as used position and further prevent folding or wrinkling.

In FIG. 10 is shown the device 10 herein, as in FIG. 9, showing the circumferential anchor point 17 formed at the intersection of the annular pliable ring portion 14 to the flexible body portion 16. Also depicted are radially extending projections 19 as well as projections 19 running traverse to the radially extending projections 19. Recesses 21 are formed on the second surface 15 therebetween.

It should be noted that any of the different depicted and described configurations and components of the thumb protector for a cast, can be employed with any other configuration or component shown and described as part of the device herein. Additionally, while the present invention has been described herein with reference to particular embodiments thereof and/or steps in the method of production or use, a latitude of modifications, various changes and substitutions are intended in the foregoing disclosure, and it will be appreciated that in some instance some features, or configurations, of the invention could be employed without a corresponding use of other features without departing from the scope of the invention as set forth in the following claims. All such changes, alternations and modifications as would occur to those skilled in the art are considered to be within the scope of this invention as broadly defined in the appended claims.

What is claimed is:

1. A thumb protector apparatus comprising:
a body formed of flexible material;
said body having a ring portion engaged to a surrounding portion;
said ring portion having a curved exterior surface extending from an engagement to said surrounding portion;
said ring portion surrounding a thumb opening therein;
said surrounding portion having a first side surface;
said surrounding portion having a second side surface opposite said first side surface;
said body positionable to an as-used position engaged with a cast located on the hand of a user wherein said first side surface faces both of two sides of a hand of said user having their thumb projecting through said thumb opening; and
said curved exterior surface of said ring portion defining a pliable pad positioned in between said thumb of said user and a hardened cast surrounding it.

2. The thumb protector apparatus of claim 1, additionally comprising:
an overhang of said curved exterior surface of said ring portion above said engagement to said surrounding portion defining an anchor point; and
said anchor point forming an anchor between said second side surface and cast fabric forming said cast which is wrapped in a contact with said second side surface.

3. The thumb protector apparatus of claim 1, additionally comprising:
a position stabilizer positioned upon said second side surface; and
said position stabilizer forming a contact with a padding layer wrapped in a compressed contact against said second side surface of said cast whereby said contact holds said body in place.

4. The thumb protector apparatus of claim 2, additionally comprising:
a position stabilizer positioned upon said second side surface; and
said position stabilizer forming a contact with a padding layer wrapped in a compressed contact against said second side surface of said cast whereby said contact holds said body in place.

5. The thumb protector apparatus of claim 3 wherein said position stabilizer comprises:
a plurality of projections rising from said second side surface; and
a plurality of recesses formed in between said plurality of projections.

6. The thumb protector apparatus of claim 4 wherein said position stabilizer comprises:
a plurality of projections rising from said second side surface; and
a plurality of recesses formed in between said plurality of projections.

7. The thumb protector apparatus of claim 5 wherein said plurality of projections rising from said second side surface extend radially around said ring portion.

8. The thumb protector apparatus of claim 6 wherein said plurality of projections rising from said second side surface extend radially around said ring portion.

9. The thumb protector apparatus of claim 1, additionally comprising:
a plurality of indentations formed into a perimeter edge of said surrounding portion.

10. The thumb protector apparatus of claim 2, additionally comprising:
a plurality of indentations formed into a perimeter edge of said surrounding portion.

11. The thumb protector apparatus of claim 3, additionally comprising:
a plurality of indentations formed into a perimeter edge of said surrounding portion.

13. The thumb protector apparatus of claim 4, additionally comprising:
a plurality of indentations formed into a perimeter edge of said surrounding portion.

14. The thumb protector apparatus of claim 5, additionally comprising:
a plurality of indentations formed into a perimeter edge of said surrounding portion.

15. The thumb protector apparatus of claim 6, additionally comprising:
a plurality of indentations formed into a perimeter edge of said surrounding portion.

16. The thumb protector apparatus of claim 7, additionally comprising:
a plurality of indentations formed into a perimeter edge of said surrounding portion.

17. The thumb protector apparatus of claim 8, additionally comprising:
a plurality of indentations formed into a perimeter edge of said surrounding portion.

18. The thumb protector apparatus of claim 1 wherein said flexible material is formed of silicone, polyethylene, or a polyurethane gel elastomer.
